(19) United States
(12) Patent Application Publication
(10) Pub. No.: US 2021/0047661 A1
(43) Pub. Date: Feb. 18, 2021

(54) SSCF PROCESS FOR SECOND GENERATION ETHANOL PRODUCTION FROM LIGNOCELLULOSIC BIOMASS AND 2G RESIDUAL BIOMASS

(71) Applicants: Indian Oil Corporation Limited, Mumbai (IN); Department Of Biotechnology, New Delhi (IN)

(72) Inventors: Ajay Kumar SHARMA, Faridabad (IN); Manas Ranjan SWAIN, Faridabad (IN); Ajit SINGH, Faridabad (IN); Anshu Shankar MATHUR, Faridabad (IN); Ravi Prakash GUPTA, Faridabad (IN); Deepak TULI, Faridabad (IN); Suresh Kumar PURI, Faridabad (IN); Sankara Sri Venkata RAMAKUMAR, Faridabad (IN)

(21) Appl. No.: 17/075,486

(22) Filed: Oct. 20, 2020

Related U.S. Application Data
(63) Continuation-in-part of application No. 16/351,045, filed on Mar. 12, 2019.

(30) Foreign Application Priority Data
Mar. 12, 2018 (IN) 201821008982

(51) Int. Cl. C12P 7/10; C12P 7/14

(52) U.S. Cl. CPC C12P 7/10; C12P 7/14

(57) ABSTRACT

The present invention relates to a process for production of ethanol from lignocellulosic biomass via modified simultaneous saccharification and co-fermentation (SSCF), wherein the process includes transferring or recirculation of residual biomass from a first batch of SSCF to a second batch of SSCF at the time of enzymatic hydrolysis and then transferring the residual biomass in part or completely from the second batch of SSCF to a third batch of SSCF process to its hydrolysis reaction step. Overall, the average ethanol yield from three conjugative batch of SSCF is 5.06% higher compare to the single batch of SSCF without adding any additional enzyme, and hydrolysis time.

### CROSS-REFERENCE TO RELATED APPLICATIONS

This U.S. patent application is a Continuation-In-Part of U.S. patent application Ser. No. 16/351,045 which was filed on Mar. 12, 2019. The present application comprises an improvement in or a modification of the invention claimed in the specification of the main patent applied for in the U.S. patent application Ser. No. 16/351,045.

### FIELD OF THE INVENTION

The present invention relates to a process for production of ethanol from lignocellulosic biomass. More particularly, present invention relates to a process for production of ethanol from a lignocellulosic biomass, wherein the process includes transferring or recirculation of residual biomass from a first batch of simultaneous saccharification and co-fermentation (SSCF) to a second batch of SSCF at the time of enzymatic hydrolysis and then transferring residual biomass in part or completely from the second batch of SSCF to a third batch of SSCF process to its hydrolysis reaction step.

### BACKGROUND OF THE INVENTION

Simultaneous Saccharification fermentation/co-fermentation (SSF/SSCF) removes sugar inhibition on enzymatic hydrolysis thus increases the hydrolysis sugar yield and reduces contamination risk. Moreover, SSF/SSCF reduces the overall reaction time and reactor volume (Kristensen et al., 2009). SSF/SSCF sacrifices the optimal conditions for both enzymatic hydrolysis and fermentation. Typically, enzymatic hydrolysis and fermentation in SSF system the temperature is kept at 37-42° C. as a compromise for better enzymatic hydrolysis and fermentation (Dien et al., 2003b). In addition, SSF/SSCF introduces a new inhibitor (ethanol) for enzymatic hydrolysis. But the inhibitory effect from ethanol is much lower compared to cellobiose or glucose (Taherzadeh & Karimi, 2007).

The major advantage of Separate Hydrolysis and fermentation/co-fermentation (SHF/SHCF) compared to SSF/SSCF is that enzymatic hydrolysis and fermentation can be carried out at their own optimal conditions (Taherzadeh & Karimi, 2007). However, enzymes during hydrolysis is easily inhibited by its end-products (sugars), especially during high solid loading enzymatic hydrolysis (Kristensen et al., 2009; Philippidis & Smith, 1995), which demands somewhat longer hydrolysis time and high enzyme loading to achieve high sugar conversions. Another problem of this process is the high risk of contamination during enzymatic hydrolysis due to the long reaction time and high sugar concentrations (Taherzadeh & Karimi, 2007). Enzymatic hydrolysis is the limiting step for SHF, which determines the overall ethanol yield (Lau & Dale, 2009). Also, conventionally, the cellulose content in residual biomass after SSCF process is used for burning for heat generation which is not environment friendly.

US2014/0227757 A1 discloses that the pretreated biomass used for experiment was derived from AFEX (ammonium fiber explosion) pretreatment. The composition of AFEX pretreated biomass is different from acid pretreated biomass. Because in AFEX pre-treatment lignin was removed (40-45%) from biomass and glucan and hemicelluloses remain intact after the pretreatment but in acid pre-treatment hemocellulase in biomass get hydrolyzed to free sugars and almost all lignin remains intact in pretreated biomass. Due to the presence of lignin in acid pretreated biomass, it is quite difficult to conduct SSCF at low dose of enzyme (2-3 FPU/TS) using conventional SSCF process. US2014/0227757 uses higher dosage of enzyme i.e., 15 fold enzyme loading considering the conversion of mg protein of enzyme to FPU loading (Zhang et al., 2015) in described process which is quite higher compare to proposed invention (2.3 FPU/TS). Higher enzyme load in the process makes the process more costly and economical unviable. In the Prior art three commercial enzymes (Ctec, Htec and pectinase) used in a different proportion for experimental purpose but in the current practice a single commercial enzyme (Ctec) was used for SSCF which makes the process simpler. The SSCF method used in main U.S. patent application Ser. No. 16/351,045 is completely different from SSCF used in the US2014/0227757. In US2014/0227757 A1 the recirculation of biomass was done at 32° C. which did not favor hydrolysis. US2014/0227757 causes the feedback inhibition in the enzymatic hydrolysis resulted to low enzymatic hydrolysis. In US2014/0227757, the reaction volume becomes more than two fold from the initial volume which makes the process unsuitable for the commercial practice.

Accordingly, present invention provides a process which overcomes the aforesaid drawback of the prior arts. In the present invention, recirculation of residual biomass was done at 50° C. which is quite favorable for the enzymatic hydrolysis. In present invention free sugars are almost absent when residual biomass added to SSCF process. In the current practice volume of the reaction mixture is increased to 20-25% when recirculation of residual biomass was done.

### SUMMARY OF THE INVENTION

Present invention relates to a process for production of ethanol from a lignocellulosic biomass, wherein the process includes recirculation of residual biomass after completion of first batch of SSCF to a second batch of SSCF at the time of enzymatic hydrolysis and then transferring half of residual biomass from the second batch of SSCF to a third batch of SSCF process to its enzymatic hydrolysis reaction step. Residual biomass from SSCF process contains residual glucan and leftover cellulase enzyme attached with residual biomass which is re-circulated to hydrolysis step (at 50° C.) of the second SSCF batch without additional requirement of enzyme for added residual biomass. Which ultimately improve the enzymatic hydrolysis resulted into higher sugar after hydrolysis thus improve final ethanol concentration in three conjugative batch SSCF. Further, in present invention of modified SSCF process, enzymatic hydrolysis is preceded by mainly C5 sugar fermentation, with low enzymatic hydrolysis and succeeds by mainly C6 sugar fermentation.

Accordingly, present invention provides a process for production of ethanol from a lignocellulosic biomass comprising;
(i) adding slurry of dilute acid pre-treated lignocellulosic biomass comprising C5 and C6 sugars in a fermenter system for first simultaneous saccharification and co-fermentation (SSCF) process;
(ii) fermenting mainly C5 along with C6 sugars by incubating the pretreated lignocellulosic biomass with a cellulase enzyme, co-fermenting microorganism and nutrient to obtain ethanol;
(iii) heating the fermenter system to 48-55° C. for a period of 18 to 24 hours for conducting a hydrolysis reaction;
(iv) allowing the fermenter system to cool to a temperature of 35-37° C.; and
(v) fermenting mainly C6 sugar and a residual C5 sugar by inoculating the fermenter system with a second dose of co-fermenting microorganism to obtain ethanol and a first residual biomass;
(vi) transferring the first residual biomass in part or completely to a second SSCF process to its hydrolysis reaction step (iii), wherein the second SSCF process comprises aforesaid steps (i) to (v) to obtain ethanol and a second residual biomass;
(vii) transferring of the residual biomass from second SSCF process in part or completely to a third SSCF process to its hydrolysis reaction step (iii), wherein the third SSCF process comprises aforesaid steps (i) to (v) to obtain ethanol.

In one of the features of the present invention the step (v) of the above process for production of ethanol from a lignocellulosic biomass wherein the first residual biomass contains some part of non-hydrolyzed glucan.

In one of the features of the present invention, the C5 sugar is selected from xylose and C6 sugar is selected from glucose.

In another features of the present invention the dilute acid pre-treated lignocellulosic biomass is obtained by a dilute acid pretreatment of the lignocellulosic biomass, which is carried out in presence of an acid at high temperature in the range of 160-190° C. which hydrolyzed xylan part into xylose sugar in majority and partially releases glucose, wherein the concentration of xylose remains higher in pretreated slurry in comparison to glucose sugar. In yet other features of the present invention, the pretreatment can occur in wide range of temperature.

In one of the feature of the present invention, the concentration of the cellulase enzyme is in the range of 2.3 to 3.3 FPU/g/TS is employed for the SSCF/fermentation process, which partially hydrolyze glucan content in the biomass and some amount of recalcitrant glucan remains in the first residual biomass, which is further transferred to two conjugative SSCF process to enhance the overall ethanol yield. Preferably, the concentration of enzyme is 2.3 FPU/gTS for the SSCF/fermentation process.

In one of the feature of the present invention, the fermentation of C5 sugar is carried out at temperature in the range of 30° C.-35° C. for 16-24 hours or any other temperature which favors fermentation over hydrolysis, when the xylose concentration is reduced to <5 g/l from 30-40 g/l in fermentation broth the temperature of process is increased to 35 to 37° C. gradually and incubated at 2 h in each temperature for better hydrolysis and fermentation simultaneously. Further temperature increased to 50° C. for enzymatic hydrolysis. In yet other feature, the xylose concentration is reduced to 3-5 g/l. The xylose concentration is reduced to <5 g/l from 30-40 g/l and depends upon solid loading of biomass.

In another feature of the present invention, the fermentation of C6 sugar is carried out at temperature in the range of 35 to 37° C. for 6 to 10 hours or any other temperature which favors fermentation over hydrolysis.

In yet another feature of the present invention, the slurry of dilute acid pre-treated biomass is added in the fermenter system of step (i) without any detoxification.

In still another feature of the present invention, the process for production of ethanol from a lignocellulosic biomass additionally comprising adjusting pH of the slurry of step (i) to 5-5.5 with a pH adjuster.

In yet another feature of the present invention, the pH adjuster is selected from aqueous NH₄OH, NaOH, KOH, and CaCO₃ or substance which is alkaline in nature and increases pH.

In still another feature of the present invention, the nutrient is ammonium sulphate, magnesium sulphate or any other magnesium or ammonium salts.

In still another feature of the present invention, the cellulase enzyme is from fungal or bacterial origin, composed of cellobiohydrolase I, II and β-glucosidase along with other accessory enzyme, wherein the other accessory enzyme is selected from xylanase, β-xyloxidase, arabinofuranosidase, and pectinse or any other enzyme which hydolyyze glucan and/or xylan.

In yet another feature of the present invention, the co-fermenting microorganism is selected from Saccharomyces cerevisiae, Pichia sp., Candida sp., and E. coli or any ethanogenic co-fermenting microorganism.

In yet another feature of the present invention, the fermentation of C6 sugar is stopped after 6 to 10 hours.

In still another feature of the present invention, optionally other nutrient is used to enhance the final ethanol concentration and the other nutrient is selected from yeast extract, peptone and ammonium sulphate or any other nitrogen source for microorganism.

In still another feature of the present invention, the lignocellulosic biomass is selected from straw, wheat straw, sugarcane bagasse, cotton stalk, barley stalk, bamboo or any agriculture residues which contain cellulose and/or hemicellulose or both.

In one of the features, present invention provides a process for production of ethanol from a lignocellulosic biomass comprising:
adding slurry of dilute acid pre-treated lignocellulosic biomass comprising C5 and C6 sugars without any detoxification in a fermenter system for first simultaneous saccharification and co-fermentation (SSCF) process;
(ii) adjusting pH of the slurry of step (i) to 5-5.5 with aqueous NH₄OH (ammonium solution) to obtain a pH adjusted slurry;
(iii) fortifying the pH adjusted slurry with magnesium sulphate (MgSO₄) in amount of 3-5 g/l, along with cellulase enzyme and co-fermenting microorganism;
(iv) adding water to the slurry of step (iii) to maintain 5 to 20 weight % Total Solid (TS) in the slurry;
(v) incubating the slurry of step (iv) at 30° C.-35° C. for 16-24 hours for mainly fermenting C5 sugars with 200-250 rpm when free xylose in the slurry to comes down to <5 g/l from 30-40 g/l of initial xylose;
(vi) heating the fermenter system to 48-55° C. at ramping of 3 to 4° C. per 20-25 minutes, and then the process is allowed to maintain the temperature 48° C.-55° C. for 18-24 hours for conducting a hydrolysis reaction of glucan and xylan;
(vii) allowing the fermenter system to cool down to a temperature in the range of 35-37° C.; and
(viii) fermenting mainly C6 sugar and a residual C5 sugar by inoculating the system with a second dose of co-fermenting microorganism to obtain ethanol and a first residual biomass;
(ix) transferring the first residual biomass in part or completely to a second SSCF process to its hydrolysis reaction step (vi), wherein the second SSCF process comprises aforesaid steps (i) to (viii) to obtain ethanol and a second residual biomass;
(x) transferring the second residual biomass from second SSCF process in part or completely to a third SSCF process to its hydrolysis reaction step (vi), wherein the third SSCF process comprises aforesaid steps (i) to (viii) to obtain ethanol.

In one of the features of the present invention, the step (v) of the above process for production of ethanol from a lignocellulosic biomass wherein the free xylose in the slurry to comes down to the <5 g/l from the 30-40 g/l initial xylose depends upon solid loading of biomass.

In one of the features of the present invention, the step (v) of the above process includes while temperatures increasing, fermenter system/fermentor is hold at 35 and 37° C. for 2 hours to increase rate of hydrolysis and fermentation.

In one of the preferred features of the present invention, the residual biomass from the first SSCF is transferred to the second batch of SSCF at time of hydrolysis process at a temperature of 50° C. for certain time limit of 24 hours.

### BRIEF DESCRIPTION OF DRAWINGS

FIG. 1 illustrates flowchart depicting a process for production of ethanol from a lignocellulosic biomass having a single batch of SSCF;

FIG. 2 illustrates results of modified SSCF using Co-fermenting S. cerevisiae and commercial enzyme (3.3 FPU/gTS) having a single batch of SSCF;

FIG. 3 illustrates results of conventional SSCF using S. cerevisiae and commercial enzyme (7 FPU/gTS);

FIG. 4 illustrates a process diagram for the ethanol production by further transferring/re-circulating the residual biomass after first SSCF to up to three conjugative batch of SSCF process.

FIG. 5 illustrates over all ethnaol production in three conjugative batch of SSCF compare to the first batch of SSCF.

### DETAILED DESCRIPTION OF THE INVENTION

While the invention is susceptible to various modifications and alternative forms, specific embodiment thereof will be described in detail below. It should be understood, however that it is not intended to limit the invention to the particular forms disclosed, but on the contrary, the invention is to cover all modifications, equivalents, and alternative falling within the scope of the invention as defined by the appended claims.

Definition:

For the purposes of this invention, the following terms will have the meaning as specified therein:

“Pre-treated biomass” or “Pretreatment of biomass” used herein clears away physical and chemical barriers that make native biomass recalcitrant and exposes cellulose for better enzymatic hydrolysis. In most of the pretreatment, chemical (acid or alkali) and physical (high temperature or pressure) parameters are used individually or in mixed manner to remove barriers for enzymatic hydrolysis and improve the enzymatic digestibility.

“Detoxification” used herein is the process where the inhibitors (toxic compound such hydroxymethyl furfural, furfural, acetic acids, formic acids etc.) produced during the pretreatment process are removed or neutralized from pre-treated biomass by chemical, physical or biological process.

“Cellulase enzyme” used herein is a mixed form of enzyme which is mostly composed of exo-hydrolase, endo-hydrolase and beta-glucosidase. This enzyme was mostly produced from fungal sources. Cellulase breaks down the cellulose molecule into monosaccharide and shorter polysaccharides or oligosaccharides. In the present invention the cellulase enzyme is selected from commercial available cellulase enzymes which are suitable for the purposes. More particularly commercial available cellulase enzyme CTec3 is used in the present invention.

“Free sugar” used herein is the monomeric form of sugar which are produced from the lignocellulosic biomass during the pretreatment. Free sugar in this process composed of mainly glucose and xylose.

“C5 sugar” used herein is represented for Xylose. “Free C5 sugar” used herein is sugar (mostly xylose) released from the hemicelluloses during the pretreatment and some part in enzymatic hydrolysis.

“C5 fermentation” used herein is Xylose fermentation into ethanol.

“C6 sugar” used herein represents glucose.

“C6 fermentation” used herein is Glucose fermentation into ethanol.

“2G residual biomass” is left over biomass after SSCF/SHF process contains lower cellulose and/or hemicelluloses than native biomass

“Nutrient” used herein is Ammonium hydroxide and MgSO₄. Ammonium hydroxide used in this process has dual activity, it adjust the pH of the sulphuric acid (H₂SO₄) pretreated biomass and simultaneously converted to ammonium sulphate (ammonium ion (NH₄⁺) combined with free sulphates (SO₄²⁻) ions released from the sulphuric acid during the pretreatment. Ammonium sulphate ((NH₄)₂SO₄) acts as a nitrogen source for yeast during fermentation. Another salt MgSO₄ used in fermentation where, Mg²⁺ act as an essential enzyme cofactor of most biological pathways. During fermentation Mg²⁺ plays a major role for proper functioning of fermenting enzymes in yeast.

In the main U.S. patent application Ser. No. 16/351,045, free form of C5 sugars in acid pretreated biomass was targeted first for the SSCF using C5 utilizing yeast at low temperature (30° C.) along with the cellulase enzyme. Further the temperature of the process is increased to 50° C. for better cellulytic hydrolysis for certain time limit (24 h) and then temperature of process is reduced to 35° C. for better fermentation of C6 sugar by adding a second dose of yeast to the fermentation broth. The whole process took 46 to 48 h to achieve 4.7 to 5% ethanol titer in the final fermentation broth at 20% solid loading of biomass. In this process the C5 and C6 sugars targeted for fermentation in sequence manner to achieve higher ethanol titer at short time of SSCF (48 h). This process is advantageous over conventional SSCF because initial free sugars in the pretreated biomass was targeted which reduces the enzymatic feedback inhibition and improve the ethanol yield with low dose of enzyme at short time interval. With this strategy, an overall ethanol yield was about 70% of the theoretical maximum both C5 and C6 sugars from the pretreated biomass. C5 utilization exceeded 95% after SSCF without formation of any byproduct (xylitol). Sequential targeting of C5 and C6 sugars conversion sustained C5 fermentation and improved C5 utilization and ethanol yield.

Simultaneous saccharification and co-fermentation (SSCF) is a promising strategy for obtaining high ethanol yield. After SSCF residual biomass left over contains significant amount of residual glucan in it which can be hydrolyzed into free glucose. The residual glucan is recalcitrant in nature to break into glucose and needs more time for the hydrolysis. In normal practice the residual biomass considered as waste after SSCF process which is subjected for burning to generate electricity or used for heating boilers in large scale operation units which ultimately induces air pollution. To overcome this issue, in the present invention, residual biomass from first SSCF is transferred to the second batch of SSCF at time of hydrolysis process at a temperature in the range of 48-55° C. and more preferably at 50° C. for certain time limit of 18-24 hours and more preferably 24 hours. The final ethanol concentration in second batch SSCF is 9% higher than first batch SSCF. Similarly, half of residual biomass of second SSCF is transferred to third batch of SSCF to enhance higher ethanol titer, which is 5.28% higher than first SSCF. The single SSCF process took 48 h to achieve 2.8 to 3% ethanol titer in the final SSCF broth at 15% final solid loading at 2.3 FPU/Total solid. In the process of the present invention, the C5 and C6 sugars targeted for SSCF in sequence manner to achieve higher ethanol titer at short time of SSCF (48 h). Overall, the average ethanol yield from three conjugative SSCF is 5.06% higher compare to the single process of SSCF without adding any additional enzyme and hydrolysis time.

Xylan and Glucan are polymer of xylose and glucose respectively collectively called as holocellulose in lignocellulosic biomass. As per the physical property xylan and glucan are amorphous and crystalline in nature respectively. Due to the physical property, xylan gets breaks down to xylose when lignocellulosic biomass subjected to acid pretreatment but most of the glucan remain un-reacted. So in this process when the pretreated biomass is taken for SSCF, free form of xylose (30-35 g/L) (breakdown xylan) are present in the biomass which is targeted firstly by the co-fermenting microorganism for SSCF in presence of very less amount of glucose (<8-10 g/L, which is released during the pretreatment) at 30-35° C. The SSCF temperature is not adequate enough for the enzyme to breakdown of the glucan to glucose efficiently. So due to this the co-fermenting microorganism mostly targeted xylose (C5) sugar at the initial stage of SSCF. Accordingly, the present invention discloses a method for production of ethanol from lignocellulosic biomass having three conjugative batch of SSCF process. In the present invention, free C5 sugar in pre-treated biomass is targeted first along with available low concentration of glucose for SSCF followed by enzymatic hydrolysis and C6 SSCF in sequential manner.

The process, in accordance with the present invention, brings the C5 concentration about to dryness and brings down the total process time (both hydrolysis and SSCF) to 46 h which is about ⅓ of the conventional SHF (total process time 120 h which include 72 h Hydrolysis and 48 h SSCF). Overall ethanol productivity is much higher than conventional SSCF process.

The main U.S. patent application Ser. No. 16/351,045 provides a method for production of ethanol from lignocellulosic biomass having a single batch of SSCF; (see FIG. 1) comprising:
1. Addition of acid pre-treated biomass slurry without any detoxification in the fermenter;
2. The pH of the slurry was adjusted to 5-5.5 with aqueous ammonium solution. The pH adjusted slurry was fortified with 3-5 g/l MgSO₄, cellulase enzyme and co-fermenting Saccharomyces cerevisiae; the co-fermenting yeast is essential to use in this process to utilize both pentose and hexose sugar in the SSCF broth.
3. Appropriate water is added in to fermenter to adjust the final Total solid (TS) loading at 20% of biomass. The whole process is then incubated at 30° C.-35° C. for 16-20 hours for the SSCF with 200 rpm;
4. When the concentration of free xylose (free xylose is produced during the process of acid pretreatment and readily available for SSCF during SSCF process) in the slurry comes down to 3-5 g/l, the temperature of the process was slowly increased to 48-55° C. at ramping of 3 to 4° C. per 20-25 min., then the process is allowed to maintain about 48-55° C. temperature for 18-24 hours. After this incubation the system was allowed to cool down to temperature 35-37° C.;
5. A second dose of co-fermenting Saccharomyces cerevisiae is inoculated to the system for the second stage of SSCF. The second SSCF was stopped after 6 to 10 hours of SSCF.

The present application comprises an improvement in or a modification of the invention claimed in the specification of the main patent applied for. Accordingly, present invention provides an improved method of simultaneous saccharification and co-fermentation (SSCF) method for second generation ethanol production from lignocellulosic biomass and recirculation of residual biomass. The process of the present invention includes recirculation of residual biomass from a first batch of SSCF to a second batch of SSCF at the time of enzymatic hydrolysis and then transferring half of residual biomass from the second batch of SSCF to a third batch of SSCF process to its hydrolysis reaction step. Overall, the average ethanol yield from three conjugative batch of SSCF is 5.06% higher compare to the single batch of SSCF without adding any additional enzyme, and hydrolysis time.

In one feature of the present invention, in this current SSCF process very less enzyme (2.3 FPU/gTS) in the SSCF process is used which partially hydrolyze glucan content in the biomass and some amount of recalcitrant glucan remains in residual biomass, which is further re-circulated to two conjugative SSCF process to enhance the overall ethanol SSCF. From the result it is observed that overall, the average 5.06% ethanol yield was enhanced in the SSCF broth compare to the earlier process. The residual biomass from previous batch of SSCF is considered as a waste and normally used for burning purposes. So, in this process the waste gets utilized in conjugative SSCF process to enhance final ethanol concentration. This improved SSCF process does not require additional SSCF time or enzyme dose for enhancement of ethanol yield which is significantly better compare to previous prior art. Another advantage of invention includes higher ethanol yield and productivity without addition of enzyme or increasing hydrolysis time of biomass.

In one feature, process of present invention is of second generation ethanol SSCF and an improved method compare to the existing prior art as it increases ethanol titer, ethanol yield and productivity even at similar total process time and enzyme loading.

In accordance with the process of present invention, the low enzyme loading (2.3 FPU/total solid loading) was used in the SSCF process which is comparatively low (34%) to the existing process. Overall, the present approach of SSCF reduces the processing cost by reducing the enzyme dose, chemicals and using less water during the process. Residual biomass of after SSCF process gets utilized by next SSCF process to enhance ethanol yield without increasing the SSCF time and enzyme load in SSCF process. Overall, the current process is economically cheaper and time saving process than the conventional SSCF process.

Having described the basic aspects of the present invention, the following non-limiting examples illustrate specific embodiment thereof.

### EXAMPLE 1

Process for Production of Ethanol from a Lignocellulosic Biomass Having a Single Batch of SSCF:

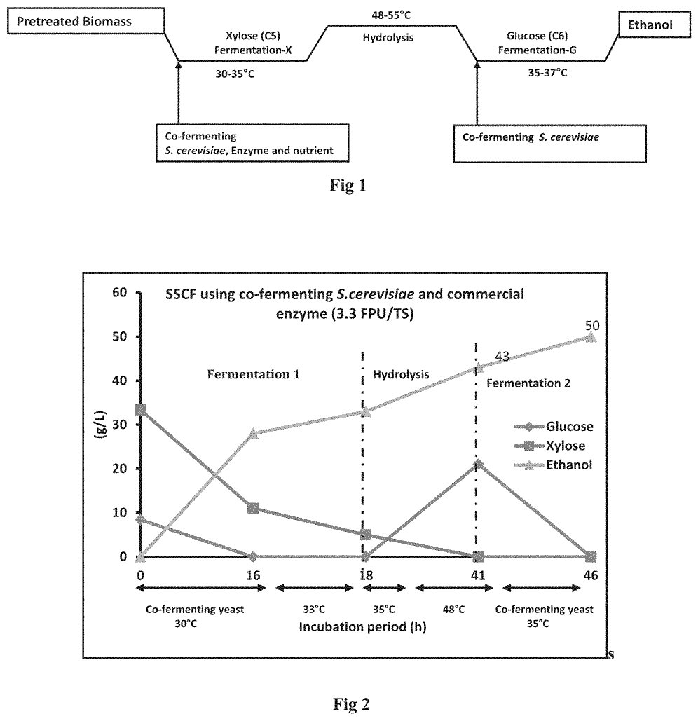

Pretreated biomass (slurry, TS approximately 24%) without any detoxification is introduced directly to the fermenter. The pH of the slurry was adjusted to 5.5 with aqueous ammonium solution (25% initial concentration). The pH adjusted slurry was fortified with 3 g/l MgSO₄, cellulase enzyme (Commercial enzyme, 3.3 FPU/gTS) and co-fermenting Saccharomyces cerevisiae (1 g dry cell biomass/liter, xylose and glucose utilizing yeast). Required amount of water was added to the process to adjust the final biomass concentration to 20%. The whole process was incubated at 30° C. for 16 h for the SSCF with 200 rpm. When the free xylose concentration in the slurry comes near to 6-7 g/l , the temperature of the process was increased to 33° C. and 35° C., incubated for 2 h in each temperature for better hydrolysis and SSCF. After that temperature increased to 48° C. This step mainly required for rapid releases of glucose sugar from cellulose which converted simultaneously with hydrolysis to ethanol by yeast biomass. As the temperature was reached at desired target the process was allowed to maintain the required temperature (48° C.) for 23 h for better enzymatic hydrolysis. After this incubation the system was allowed to cool down to temperature 35° C. A second dose of co-fermenting S. cerevisiae (1 g dry cell biomass/liter) was inoculated to the system for the second stage of fermentation. The second fermentation was stopped after 6 h of fermentation. This process took 46 h incubation including fermentation and enzymatic hydrolysis. The results of this experiment are represented by FIG. 2.

| | |
|---|---|
| Solid Loading in SSCF | 20% |
| Mode of SSCF | SSCF, Single yeast |
| | strain co-fermenting |
| | Saccharomyces cerevisiae |
| | (2 g/L) used in |
| | both SSCF |
| Enzyme loading (FPU/g) and Sources | 3.3, Commercial enzyme |
| Residual Xylose (g/L) | 1.30 |
| Ethanol Concentration (g/L) at 46 h | 50 |
| Ethanol Yield (%) | 71 |
| Specific Productivity (Q) g/L/h | 1.08 |

### EXAMPLE 2

Conventional SSCF Process for Ethanol Production:

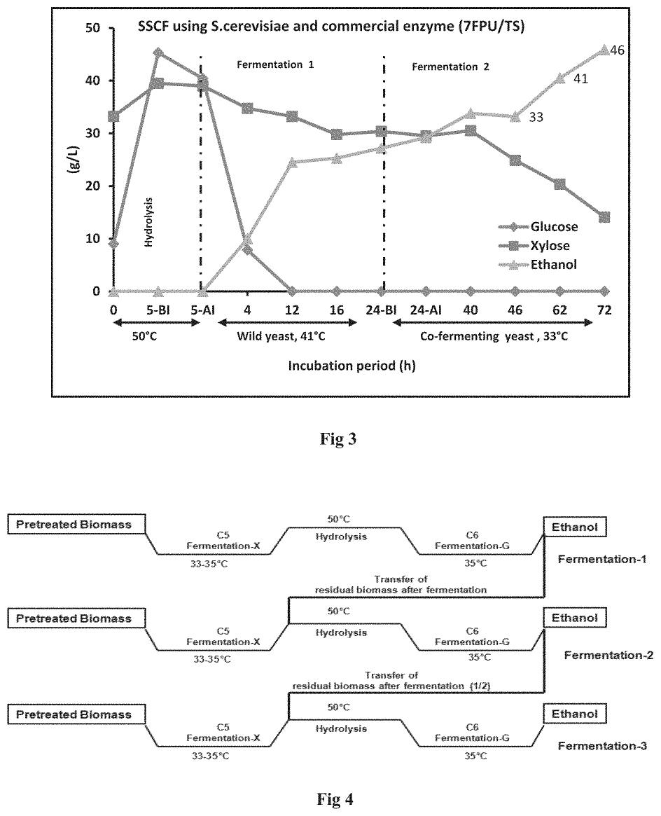

Using conventional SSCF approach of ethanol production from pretreated biomass, saccharification at 50° C. for 5 h and followed by fermentation and hydrolysis at 41° C. by a moderately thermo tolerant wild yeast S. cerevisiae up to 24 h. After this fermentation another yeast co-fermenting S. cerevisiae was inoculated to the SSCF process. In this approach the xylose utilization after the glucose SSCF was comparatively slow as compare to the above process and about 10 g/l residual xylose was observed after 72 h. This process of fermentation brings the lower ethanol titer after the 72 h of fermentation using even higher enzyme dosage. The results of this experiment are represented by FIG. 3.

| | |
|---|---|
| Solid Loading in SSCF | 20% |
| Mode of SSCF | SSCF, 1^{st} wild type |
| | Saccharomyces cerevisiae (1 g/l), |
| | 2^{nd} co-fermenting |
| | Saccharomyces cerevisiae (1 g/l) |
| Enzyme loading (FPU/g) and Sources | 7, Commercial enzyme |
| Residual Xylose (g/L) | 9.98 |
| Ethanol Concentration (g/L) at 72 h | 46 |
| Ethanol Yield (%) | 65 |
| Specific Productivity (Q) g/L/h | 0.64 |

### EXAMPLE 3

Process for the Ethanol Production By Transferring/Re-Circulating the Residual Biomass After First SSCF to Up to Three Conjugative Batch of SSCF Process:

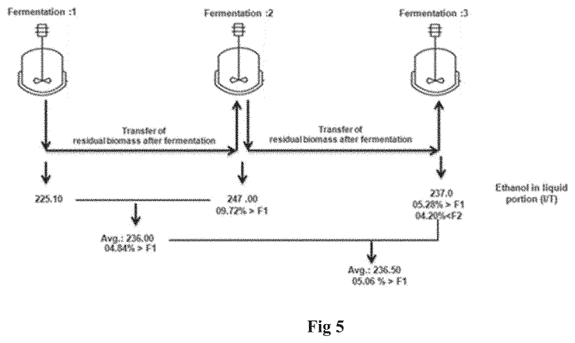

Present invention reveals a novel process of SSCF which achieves 2.8 to 3% ethanol titer from 15% dilute acid pretreated rice straw within 48 h of SSCF. The significant of the process is that after pretreatment the pretreated biomass (slurry, TS approximately 20%) without any detoxification comes directly to the fermentor. The pH of the slurry was adjusted to 5-5.5 with aqueous ammonium solution (25% initial concentration). The pH adjusted slurry was fortified with MgSO₄ (0.5%), cellulase enzyme (commercial enzyme, 2.3 FPU/gTS) and engineered co-fermenting Saccharomyces cerevisiae (1 g dry cell biomass/100 gTS, xylose utilizing genetically modified yeast). Required amount of water was added to the process to maintain the final biomass concentration to 15%. The whole process was incubated at 33° C. for 18 h for the fermentation with 200 rpm. When the free xylose concentration in the slurry comes down to approximately 3 g/l, the temperature of the process was slowly increased to 50° C. at ramping of 3 to 4° C./20 min. After the temperature was reached at desired target the process was allowed to maintain the temperature for 22 h for better enzymatic hydrolysis. After this incubation the system was allowed to cool down to temperature 35° C. After which another dose of co-fermenting S. cerevisiae (1 g dry cell biomass/100 gTS) was inoculated to the system for the second stage of fermentation. The second stage of fermentation was stopped after 6 to 8 h of fermentation. As a whole, the process took 48 h incubation including fermentation and enzymatic hydrolysis. After the first batch of SSCF the solid and liquid was separated and the solid portion was transferred to the conjugative second batch of SSCF at the time of hydrolysis by keeping the percussion that the residual ethanol from the first batch should not transfer to the next batch of SSCF. Then transferred the residual biomass from second batch of SSCF process which is about half of initial biomass at solid loading for next third batch of SSCF process. The biomass transferred to SSCF process and ethanol production from this combined process are illustrated in FIGS. 4 and 5 respectively.

TABLE A
Parameters used for the SSCF process for
the ethanol production by recalculating the
residual biomass after first SSCF to up
to three conjugative batch of SSCF process

| | |
|---|---|
| Solid Loading | 15% |
| Yeast biomass | 1.0 g dry cell |
| | biomass/Lit |
| Enzyme loading (FPU/g) and | 2.3, Commercial |
| Sources | enzyme |
| Ethanol Concentration (g/L) | 33 |
| (average of three conjugative | |
| fermentation) | |
| Specific Productivity (Q) g/L/h | 0.71 |

In present invention, simultaneous saccharification and co-fermentation is considered as the advantageous over conventional SSCF due to several regions as follows.
Higher solid loading (15%) at initial stage of SSCF.
Low enzyme loading (2.3 to 3.3 FPU/total solid)
Less SSCF process time (48 h) to achieve higher ethanol titer
Low cost nutrient sources [Urea (0.2%), MgSO₄ (0.2%)]
Less water requirement in the process of SSCF.
Un-sterilized whole acid-pretreated biomass without any detoxification
Low CAPEX and OPEX

### REFERENCES

1. Krishnan, C., Sousa, L. D., Jin, M. J., Chang, L. P., Dale, B. E., Balan, V. 2010. Alkali-based AFEX Pretreatment for the conversion of sugarcane bagasse and cane leaf residues to ethanol. Biotechnology and Bioengineering, 107(3), 441-450.
2. Dien, B. S., Cotta, M. A., Jeffries, T. W. 2003. Bacteria engineered for fuel ethanol production: current status. Applied Microbiology and Biotechnology, 63(3), 258-266.
3. Taherzadeh, M. J., Karimi, K. 2007. Enzyme-based hydrolysis processes for ethanol from lignocellulosic materials: a review. Bioresources, 2(4), 707-738.
4. Lau, M. W., Dale, B. E. 2009. Cellulosic ethanol production from AFEX-treated corn stover using Saccharomyces cerevisiae 424A (LNH-ST). Proceedings of the National Academy of Sciences of the United States of America, 106(5), 1368-1373.

1. A process for production of ethanol from a lignocellulosic biomass comprising:
adding slurry of dilute acid pre-treated lignocellulosic biomass comprising C5 and C6 sugars in a fermenter system for first simultaneous saccharification and co-fermentation (SSCF) process;
(ii) fermenting mainly C5 along with C6 sugars by incubating the pretreated lignocellulosic biomass with a cellulase enzyme, co-fermenting microorganism and nutrient to obtain ethanol;
(iii) heating the fermenter system to 48-55° C. for a period of 18 to 24 hours for conducting a hydrolysis reaction;
(iv) allowing the fermenter system to cool to a temperature of 35-37° C.; and
(v) fermenting mainly C6 sugar and a residual C5 sugar by inoculating the fermenter system with a second dose of co-fermenting microorganism to obtain ethanol and a first residual biomass;
(vi) transferring the first residual biomass in part or completely to a second SSCF process to its hydrolysis reaction step (iii), wherein the second SSCF process comprises aforesaid steps (i) to (v) to obtain ethanol and a second residual biomass;
(vii) transferring of the residual biomass from second SSCF process in part or completely to a third SSCF process to its hydrolysis reaction step (iii), wherein the third SSCF process comprises aforesaid steps (i) to (v) to obtain ethanol.

2. The process as claimed in claim 1, wherein the C5 sugar is selected from xylose and C6 sugar is selected from glucose.

3. The process as claimed in claim 1, wherein the dilute acid pre-treated lignocellulosic biomass is obtained by a dilute acid pretreatment of the lignocellulosic biomass, which is carried out in presence of an acid at high temperature in the range of 160-190° C. which hydrolyzed xylan part into xylose sugar in majority and partially releases glucose, wherein the concentration of xylose remains higher in pretreated slurry in comparison to glucose sugar.

4. The process as claimed in claim 1, wherein the concentration of the cellulase enzyme is in the range of 2.3 to 3.3 FPU/g/TS is employed for the fermentation process, which partially hydrolyze glucan content in the biomass and some amount of recalcitrant glucan remains in the first residual biomass, which is further transferred to two conjugative SSCF process to enhance the overall ethanol yield.

5. The process as claimed in claim 1, wherein the fermentation of C5 sugar is carried out at temperature in the range of 30° C.-35° C. for 16-24 hours or any other temperature which favors fermentation over hydrolysis, when the xylose concentration is reduced to <5 g/l from 30-40 g/l in fermentation broth the temperature of process is increased to 35 to 37° C. gradually and incubated at 2 h in each temperature for better hydrolysis and fermentation simultaneously.

6. The process as claimed in claim 1, wherein the fermentation of C6 sugar is carried out at temperature in the range of 35 to 37° C. for 6 to 10 hours or any other temperature which favors fermentation over hydrolysis.

7. The process as claimed in claim 1, wherein the slurry of dilute acid pre-treated biomass is added in the fermenter system of step (i) without any detoxification.

8. The process as claimed in claim 1, additionally comprising adjusting pH of the slurry of step (i) to 5-5.5 with a pH adjuster.

9. The process as claimed in claim 8, wherein the pH adjuster is selected from aqueous NH₄OH, NaOH, KOH, and CaCO₃ or substance which is alkaline in nature and increases pH.

10. The process as claimed in claim 1, wherein the nutrient is ammonium sulphate, magnesium sulphate or any other magnesium or ammonium salts.

11. The process as claimed in claim 1, wherein the cellulase enzyme is from fungal or bacterial origin, composed of cellobiohydrolase I, II and β-glucosidase along with other accessory enzyme, wherein the other accessory enzyme is selected from xylanase, β-xyloxidase, arabinofuranosidase, and pectinse or any other enzyme which hydolyyze glucan and/or xylan.

12. The process as claimed in claim 1, wherein the co-fermenting microorganism is selected from Saccharomyces cerevisiae, Pichia sp., Candida sp., and E. coli or any ethanogenic co-fermenting microorganism.

13. The process as claimed in claim 1, wherein the lignocellulosic biomass is selected from straw, wheat straw, sugarcane bagasse, cotton stalk, barley stalk, bamboo or any agriculture residues which contain cellulose and/or hemicellulose or both.

14. A process for production of ethanol from a lignocellulosic biomass comprising:
adding slurry of dilute acid pre-treated lignocellulosic biomass comprising C5 and C6 sugars without any detoxification in a fermenter system for first simultaneous saccharification and co-fermentation (SSCF) process;
(ii) adjusting pH of the slurry of step (i) to 5-5.5 with aqueous NH₄OH to obtain a pH adjusted slurry;
(iii) fortifying the pH adjusted slurry with magnesium sulphate in amount of 3-5 g/l, along with cellulase enzyme and co-fermenting microorganism;
(iv) adding water to the slurry of step (iii) to maintain 5 to 20 weight % Total Solid (TS) in the slurry;
(v) incubating the slurry of step (iv) at 30° C.-35° C. for 16-24 hours for mainly fermenting C5 sugars with 200-250 rpm when free xylose in the slurry to comes down to <5 g/l from 30-40 g/l of initial xylose;
(vi) heating the fermenter system to 48-55° C. at ramping of 3 to 4° C. per 20-25 minutes, and then the process is allowed to maintain the temperature 48° C.-55° C. for 18-24 hours for conducting a hydrolysis reaction of glucan and xylan;
(vii) allowing the fermenter system to cool down to a temperature in the range of 35-37° C.; and
(viii) fermenting mainly C6 sugar and a residual C5 sugar by inoculating the system with a second dose of co-fermenting microorganism to obtain ethanol and a first residual biomass;
(ix) transferring the first residual biomass in part or completely to a second SSCF process to its hydrolysis reaction step (vi), wherein the second SSCF process comprises aforesaid steps (i) to (viii) to obtain ethanol and a second residual biomass;
(x) transferring the second residual biomass from second SSCF process in part or completely to a third SSCF process to its hydrolysis reaction step (vi), wherein the third SSCF process comprises aforesaid steps (i) to (viii) to obtain ethanol.
